(19) United States
(12) Patent Application Publication
(10) Pub. No.: US 2003/0050478 A1
(43) Pub. Date: Mar. 13, 2003

(54) Process for the continuous preparation of substituted oxazoles

(76) Inventors: Harald Rust, Neustadt (DE); Kirsten Burkart, Ludwigshafen (DE); Tillmann Faust, Weisenheim (DE); Jochem Henkelmann, Mannheim (DE); Alois Kindler, Grunstadt (DE); Christian Knoll, Neuhofen (DE); Andre Mohry, Wesseling (DE)

Correspondence Address: Herbert B. Keil, KEIL & WEINKAUF, 1350 Connecticut Ave., N.W., Washington, DC, 20036, US

(21) Appl. No.: 10/207,894

(22) Filed: Jul. 31, 2002

(30) Foreign Application Priority Data
Aug. 3, 2001 (DE) 10137627.8
Mar. 5, 2002 (DE) 10209447.0

(51) Int. Cl. C07D 263/02

(52) U.S. Cl. 548/228

(57) ABSTRACT

The present invention relates to a process for the continuous preparation of 5-alkoxy-substituted oxazoles, in particular for the continuous preparation of 4-methyl-5-alkoxy-substituted oxazoles, and to a process for preparing pyridoxine derivatives.

The present invention relates to a process for the continuous preparation of 5-alkoxy-substituted oxazoles, in particular for the continuous preparation of 4-methyl-5-alkoxy-substituted oxazoles, and to a process for preparing pyridoxine derivatives.

5-Alkoxy-substituted oxazoles are valuable synthons in organic chemistry. 4-Methyl-5-alkoxy-substituted oxazoles have particular significance as important precursors for the synthesis and industrial production of vitamin B₆ (Turchi et al., Chem. Rev. 1975, 75, 416).

A process which is economic and can be carried out on a large scale for preparing 5-alkoxy-substituted oxazoles, in particular 4-methyl-5-alkoxy-substituted oxazoles, is therefore of great significance.

It is known to convert α-isocyanoalkanoic esters discontinuously by thermal isomerization into the corresponding 5-alkoxy-substituted oxazoles.

Itov et al., Khimiko-Farmatsevticheskii Zhurnal, 1978, 12, 102 to 106 and Mishchenlo et al., Khimiko-Farmatsevticheskii Zhurnal, 1988, 7, 856 to 860, describe a discontinuous thermal cyclization of α-isocyanopropionic esters to give the corresponding 4-methyl-5-alkoxy-substituted oxazoles at 135° C. The yields of 4-methyl-5-alkoxy-substituted oxazoles achieved by use of various solvents are from 4 to 36%. The process has the disadvantage of low selectivity and thus the disadvantage that large amounts of by-products are produced. The commonest by-products of this reaction are the unreacted precursor (yield: 33 to 55%) and the rearranged α-cyanopropionic ester (yield 1 to 39%).

Maeda et al., Bull. Chem. Soc. Japan, 1971, 44, 1407 to 1410 disclose a discontinuous thermal cyclization of various α-isocyanocarboxylic esters to give the corresponding 5-alkoxy-substituted oxazoles at temperatures of from 150 to 180° C. Yields of 5.1 to 28.2% are reached, depending on the substituents.

JP 54-20493 describes a discontinuous process for preparing 4-methyl-5-alkoxy-substituted oxazoles by thermal cyclization of α-isocyanopropionic ester at temperatures of 155 and 170° C. in the presence of a tertiary amine. Although improved selectivities for the desired oxazoles are achieved in this case (34 to 91.5%), the low conversion (11.1 to 49.4%) leads to yields which are still unsatisfactory.

All the prior art processes have the disadvantage of low conversions and low selectivities and thus low yields of 5-alkoxy-substituted oxazoles. Because of the discontinuous procedure, the space-time yields in the prior art processes are only low.

It is an object of the present invention to provide a further process for preparing 5-alkoxy-substituted oxazoles with advantageous properties which no longer have the disadvantages of the prior art and provides the 5-alkoxy-substituted oxazoles in high yields and high space-time yields.

We have found that this object is achieved by a process for the continuous preparation of 5-alkoxy-substituted oxazoles of the formula I

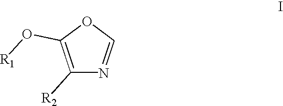

where

R₁ is an optionally substituted C₁-C₆-alkyl radical and

R₂ is hydrogen or an optionally substituted C₁-C₆-alkyl radical, by converting α-isocyanoalkanoic esters of the formula II

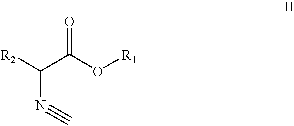

which are fed in continuously in the presence of bases at temperatures above 80° C. in a reactor into the 5-alkoxy-substituted oxazoles of the formula I and discharging the reaction products continuously from the reactor.

An optionally substituted C₁-C₆-alkyl radical means for the radicals R₁ and R₂ independently of one another branched or unbranched, optionally substituted C₁-C₆-alkyl radicals such as, for example, optionally substituted methyl, ethyl, propyl, 1-methylethyl, n-butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl.

The nature of the substituents is not critical. The C₁-C₆-alkyl radicals may, depending of the free bonding possibilities, contain up to 6 substituents, preferably selected from the group of aryl, hydroxyaryl, —NO₂, —NH₂, —OH, —CN, —COOH, or halogen, in particular F or Cl.

In a preferred embodiment, the C₁-C₆-alkyl radicals of the radicals R₁ and R₂ are unsubstituted.

Preferred radicals for R₁ are C₁-C₄-alkyl radicals such as, for example, methyl, ethyl, isopropyl, n-propyl, n-butyl, sec-butyl or tert-butyl, particularly preferably n-butyl.

Preferred radicals for R₂ are hydrogen and C₁-C₄-alkyl radicals such as, for example, methyl, ethyl, isopropyl, n-propyl, n-butyl, sec-butyl or tert-butyl, particularly preferably methyl.

Combination of the preferred radicals for R₁ and R₂ is preferred, and the combination R₁=n-butyl and R₂=methyl is particularly preferably preferred.

In a particularly preferred embodiment of the process of the invention, accordingly, n-butyl α-isocyanopropionate is converted into 4-methyl-5-n-butoxyoxazole.

The α-isocyanoalkanoic esters of the formula II used in the process of the invention can be employed in any purity.

The α-isocyanoalkanoic esters of the formula II can be prepared in a manner known per se from the corresponding formamido acid esters of the formula V

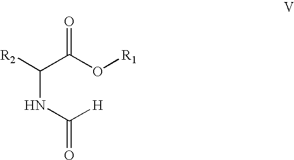

by reaction with phosphorus oxychloride or phosgene in the presence of bases. Customary synthetic methods are described in Itov et al., Khimiko-Farmatsevticheskii Zhurnal, 1978, 12, 102-106; Maeda et al., Bull. Chem. Soc. Japan, 1971, 44, 1407-1410; Ugi et al., Chem. Ber. 1961, 94, 2814; Chem. Ber. 1960, 93, 239-248, Angew. Chem. 1965, 77, 492-504, Chem. Ber. 1975, 1580-1590, DE 30 29 231 A1 and J. Heterocyclic Chemistry 1988, 17, 705.

Bases in the process of the invention mean compounds with Brönsted base properties. Preferred bases are tertiary amines such as, for example, triethylamine, triisopropylamine, tri-n-butylamine, dimethylcyclohexylamine, tris(2-ethylhexyl)amine, N-methylpyrrolidone, N,N,N′,N′-tetramethyl-1,3-propanediamine, N,N-diethylaniline or N,N-dibutylaniline. The use of tri-n-butylamine as base is particularly preferred.

Negligible thermal cyclization takes place below 80° C. The temperature for the conversion of the invention is therefore at least 80° C.

In a preferred embodiment, the process of the invention takes place at temperatures of from 100 to 200° C., particularly preferably at temperatures of from 120 to 170° C., very particularly preferably at temperatures of from 130 to 170° C.

In the process of the invention, the α-isocyanoalkanoic esters of the formula II and the bases are fed continuously, either as mixture or separately, into a reactor, the α-isocyanoalkanoic esters of the formula II are converted in the reactor into the 5-alkoxy-substituted oxazoles of the formula I, and subsequently the reaction products are removed continuously from the reactor.

The molar ratio of base to α-isocyanoalkanoic ester of the formula II is not critical and is preferably from 10:1 to 0.05:1.

The process of the invention can be carried out particularly advantageously by removing the 5-alkoxy-substituted oxazoles of the formula I from the reaction mixture during the conversion in the reactor, that is to say simultaneously with the conversion. This removal likewise preferably takes place continuously.

There are many designs of reactors which are suitable for the preferred process of the invention. Preferred reactors ought to have the property of enabling continuous conversion with simultaneous removal of a reaction product.

Examples of reactors which can be used are boilers with fitted column, extraction columns, bubble-cap tray columns, membrane reactors, Lord reactors or reaction columns.

As well known to the skilled worker, the term column means, unless mentioned otherwise, a column structure with bottom.

A fitted column accordingly means only the column structure without bottom.

Reaction columns preferably mean columns whose internals exhibit a hold-up, i.e. for example columns with plates, random packings, ordered packings or structured packings.

In a particularly preferred embodiment of the process of the invention, the conversion takes place in a reaction column as reactor.

The design and internals of the reaction columns can be as desired. It is particularly preferred to use a dividing wall, as reaction column.

A reaction column, which can have a wide variety of designs, has the property as reactor of enabling simultaneous conversion of reactants and removal of the 5-alkoxy-substituted oxazoles of the formula I from the reaction mixture by rectification.

In this preferred embodiment using a reaction column, it is further advantageous to adjust the rectification parameters so that

A the conversion of the α-isocyanoalkanoic esters of the formula II into the 5-alkoxy-substituted oxazoles of the formula I takes place on the internals and, where appropriate in the bottom of the reaction column,

B the 5-alkoxy-substituted oxazoles of the formula I produced in the conversion are removed continuously with the overhead stream or side stream of the reaction column and

C the base, and the high boilers produced where appropriate in the conversion, are removed continuously and independently of one another with the bottom stream or side stream of the reaction column.

This is achieved by various settings of the rectification parameters depending on the design of the reaction column and the reactants used. Examples of suitable rectification parameters are temperature, pressure, reflux ratio in the column, design of the column and its internals, heat management and hold-up time, especially in the bottom, or energy input, which can be optimized by the skilled worker through routine tests so that features A, B and C are achieved.

In feature C, the base can, in particular, also be removed separately from the high boilers in a second side stream.

Side stream means according to the invention the continuous discharge of a substance via a side offtake of the column.

In the process of the invention, the column overhead pressure is adjusted so that the temperature in the bottom and on the internals is at least 80° C., preferably 100 to 200° C., particularly preferably 120 to 170° C.

The column overhead pressure is typically adjusted to 5 to 800 mbar so that the bottom pressure resulting therefrom is, depending on the type of column used and, where appropriate, types of column internals used, typically 5 mbar to atmospheric pressure.

The hold-up time in the reaction column is typically between 10 minutes and 7 hours, preferably between 30 minutes and 4 hours.

It is possible that the 5-alkoxy-substituted oxazoles of the formula I form an azeotropic mixture with the bases used, so that the 5-alkoxy-substituted oxazoles of the formula I can be removed as azeotropic mixture via the overhead stream.

It is advantageous in this case for the overhead pressure, and thus also automatically the bottom pressure in the column, to be adjusted, depending on the 5-alkoxy-substituted oxazole of the formula I prepared and the base used, so that the proportion of base in the azeotrope in the overhead stream is minimized.

Removal of the base from the overhead stream azeotrope takes place in this case in a manner known per se, for example by a subsequent second rectification using a different pressure (two-pressure distillation).

For example, the 4-methyl-5-n-butoxyoxazole prepared by the process of the invention forms an azeotrope with the base tri-n-butylamine. When the overhead pressure is set at 100 mbar, the azeotrope in the overhead stream is composed of 91% by weight 4-methyl-5-n-butoxyoxazole and 9% by weight tri-n-butylamine.

Removal of tri-n-butylamine from the overhead stream azeotrope can in this case take place, for example, by subsequent second rectification with an overhead pressure of 10 mbar.

The process of the invention can be carried out in the presence or absence of solvents. In a preferred embodiment, the process of the invention takes place without solvents.

In another preferred embodiment, the process of the invention takes place in the presence of an inert solvent. An inert solvent preferably means nonpolar and polar aprotic solvents such as toluene, xylene or chlorobenzene, dichloromethane, dichloroethane, dichlorobenzene, ethylene carbonate, propylene carbonate, especially chlorobenzene.

In the case where a solvent is used, it is possible for the solvent to be fed for example with the base and the α-isocyanoalkanoic ester of the formula II in a mixture or for each individual component to be fed separately and continuously into the column.

In the case where an inert solvent is used in the process of the invention, the rectification parameters are preferably adjusted so that

A the conversion of the α-isocyanoalkanoic esters of the formula II into the 5-alkoxy-substituted oxazoles of the formula I takes place on the internals and, where appropriate in the bottom of the reaction column,

B1 in the case where the solvent has a higher boiling point than the 5-alkoxy-substituted oxazoles of the formula I produced in the conversion, the 5-alkoxy-substituted oxazoles of the formula I are removed continuously with the overhead stream, and the solvent is removed continuously via the side stream or bottom stream of the reaction column, and

B2 in the case where the solvent has a lower boiling point than the 5-alkoxy-substituted oxazoles of the formula I produced in the conversion, the 5-alkoxy-substituted oxazoles of the formula I are removed continuously with a side stream, and the solvent is removed continuously with the overhead stream of the reaction column, and

C the base, and the high boilers produced where appropriate in the conversion, are removed continuously and independently of one another with the bottom stream or side stream of the reaction column.

Any embodiments of internals can be used in the reaction column, such as, for example, column trays, random packings, ordered packings or structured packings.

Particularly advantageous column trays enable the hold-up time of the liquid to be long, the preferred hold-up time on the internals of the reaction column being at least 30 min.

Preferred column trays are, for example, valve trays, preferably bubble-cap trays or related designs such as, for example, tunnel trays, Lord reactors or other internals or Thorman trays.

Preferred structured packings are, for example, structured packings of the type Mellapack® (from Sulzer), BY® (from Sulzer), B1® (from Montz) or A3® (from Montz) or packings with comparable designs.

The process of the invention has the following advantages compared with the prior art:

The process of the invention achieves selectivities of more than 95% based on the α-isocyanoalkanoic ester of the formula II employed.

The conversion is almost 100%, so that the yield of 5-alkoxy-substituted oxazoles of the formula I is more than 95% based on the α-isocyanoalkanoic ester of the formula II employed.

The continuous procedure is a further advantage of the process. The space-time yield is distinctly greater than with previously disclosed processes.

The process of the invention represents a novel and advantageous contributory synthetic step in the process for preparing pyridoxine derivatives of the formula IX

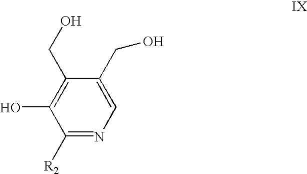

in particular for preparing pyridoxine (vitamin B₆; formula IX, R₂=methyl).

The invention therefore also relates to a process for preparing pyridoxine derivatives of the formula IX by converting amino acids of the formula III

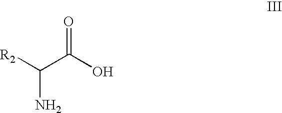

into amino acids of the formula IV,

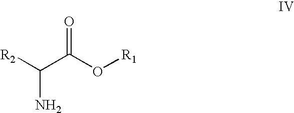

converting the latter into formamido acid esters of the formula V,

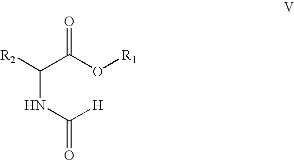

converting the latter into α-isocyanoalkanoic esters of the formula II,

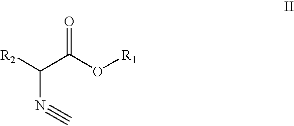

converting the latter in a continuous process step in the presence of bases at temperatures above 80° C. into 5-alkoxy-substituted oxazoles of the formula I,

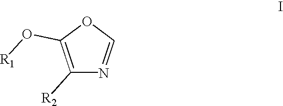

reacting the latter with protected diols of the formula VI,

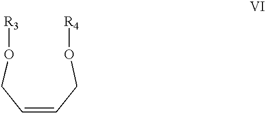

where

R₃, R₄ are, independently of one another, or R₃ and R₄ together, are a protective group of the hydroxyl function,

to give the Diels-Alder adducts of the formula VII

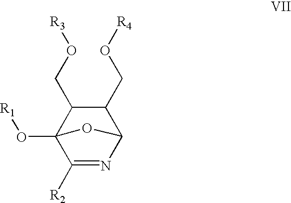

and converting the latter by acid treatment and elimination of the protective group into the pyridoxine derivatives of the formula IX.

Apart from the novel, advantageous contributory step of the continuous conversion according to the invention of α-isocyanoalkanoic esters of the formula II into 5-alkoxy-substituted oxazoles of the formula I, the overall process is disclosed in Ullmann's Encyclopedia of Industrial Chemistry 1996, Vol. A 27, pages 533 to 537.

Starting materials for the overall synthesis are low-cost amino acids of the formula III, preferably alanine (R₂=methyl). These are converted in a manner known per se, for example by acid-catalyzed esterification with alcohols R₁—OH, preferably n-butanol, into amino acid esters of the formula IV. This esterification can, however, also be achieved by other methods such as, for example, by activation of the acidic function and base-catalyzed esterification. Further methods are described in U.S. Pat. No. 3,227,721.

The amino acid esters of the formula IV are converted in a manner known per se, for example as described in U.S. Pat. No. 3,227,721, into the formamido acid esters of the formula V.

The formamido acid esters of the formula V are subsequently converted in a manner known per se, as described above, into the α-isocyanoalkanoic esters of the formula II.

The α-isocyanoalkanoic esters of the formula II are converted as described above by the process of the invention continuously into 5-alkoxy-substituted oxazoles of the formula I.

This contributory step in the preferred overall process is carried out in the preferred embodiments as described above.

The 5-alkoxy-substituted oxazoles of the formula I are then reacted with protected diols of the formula VI to give the Diels-Alder adducts of the formula VII.

This contributory step may take place after the process of the invention, but may also take place simultaneously with the conversion of the α-isocyanoalkanoic esters of the formula II into the 5-alkoxy-substituted oxazoles of the formula I by continuously feeding the protected diols of the formula VI into the reactor of the process of the invention. They are fed either mixed with the α-isocyanoalkanoic ester of the formula II, the base and, where appropriate, the solvent, or as separate components. In this case, the 5-alkoxy-substituted oxazoles are removed as product directly in the form of their Diels-Alder adduct via the bottom offtake of the column.

The radicals R₃, R₄ mean, independently of one another, a protective group, preferably an acid-labile protective group or the hydroxyl function.

It is possible in principle to use any acid-labile protective group. Preferred acid-labile protective groups are the acid-labile protective groups disclosed in the literature for hydroxyl groups (T. W. Greene, Protective Groups in Organic Synthesis, John Wiley & Sons New York, 1981, pages 14-71; P. J. Kocienski, Protecting Groups, Georg Thieme Verlag Stuttgart, 1994, pages 21-94).

A further possibility in a preferred embodiment is for the radicals R₃ and R₄ together to form an acid-labile protective group for both hydroxyl functions. For this purpose, the two hydroxyl functions preferably form a cyclic acetal with ketones or aldehydes, such as, for example, acetone or isobutyraldehyde.

Subsequent acid treatment of the Diels-Alder adducts of the formula VII results, with elimination of the alcohol R₁—OH, in aromatization to give the pyridoxine framework. Elimination of the acid-labile protective group(s), which normally takes place by treatment with aqueous acid, affords the Pyridoxine derivatives of the formula IX, in particular pyridoxine (vitamin B6, R₂=methyl).

The alcohol R₁—OH and the protective groups R₃ and R₄ can be recovered and reused in the overall process.

The use of the novel advantageous contributory step of the invention in the overall process leads to an increase in the overall yield.

The following examples illustrate the invention.

### EXAMPLE 1

Continuous preparation of 4-methyl-5-n-butoxyoxazole in a dividing wall column

A mixture of 20.5% by weight n-butyl α-isocyanopropionate (R₁=n-butyl, R₂=methyl) and 79.5% by weight tri-n-butylamine was passed into a continuously operated dividing wall column (4.8 m×64 mm) packed with 3×3 mm stainless steel Raschig rings and a dividing wall 2.4 m high with 60 theoretical plates.

With an overhead pressure of 500 mbar and a bottom temperature of 165° C., 4-methyl-5-n-butoxyoxazole distills as an azeotrope with tri-n-butylamine (90:10% by weight) at a boiling point of 158° C. High boilers and tributylamine are taken off at the bottom of the column. The conversion was 98.4%, and the selectivity was 99%. The yield of 4-methyl-5-n-butoxyoxazole 95% based on the n-butyl α-isocyanopropionate employed.

The azeotrope was subsequently separated in the same column at an overhead pressure of 10 mbar. The overhead product is an azeotrope with the composition 4-methyl-5-n-butoxyoxazole:tri-n-butylamine=70:30, and pure 4-methyl-5-n-butoxyoxazole with a boiling point of 98° C. is obtained in the side offtake. The distillation yield was 99% (40% pure 4-methyl-5-n-butoxyoxazole and 60% 4-methyl-5-n-butoxyoxazole as azeotrope which was returned to the first distillation). The purity of the 4-methyl-5-n-butoxyoxazole was 99.8%.

### EXAMPLE 2

Continuous preparation of 4-methyl-5-n-butoxyoxazole in a dividing wall column with solvent

A mixture of 13.1% by weight n-butyl α-isocyanopropionate (R₁=n-butyl, R₂=methyl), 32.2% by weight monochlorobenzene and 50.1% by weight tri-n-butylamine was passed into a continuously operated dividing wall column (4.8 m×64 mm) packed with 3×3 mm stainless steel Raschig rings and a dividing wall 2.4 m high with 60 theoretical plates.

With an overhead pressure of 300 mbar and a bottom temperature of 169° C., monochlorobenzene distills at a boiling point of 90° C., and 4-methyl-5-n-butoxyoxazole is obtained as azeotrope with tri-n-butylamine (88:12% by weight) with a boiling point of 151° C. in the side offtake. High boilers and tributylamine are taken off in the bottom of the column. The conversion was 99.5%, and the selectivity was 99%. The yield of 4-methyl-5-n-butoxyoxazole was 94% based on the n-butyl α-isocyanopropionate employed.

The azeotrope was separated in analogy to Example 1.

### EXAMPLE 3

Continuous preparation of 4-methyl-5-n-butoxyoxazole in a reaction column with solvent

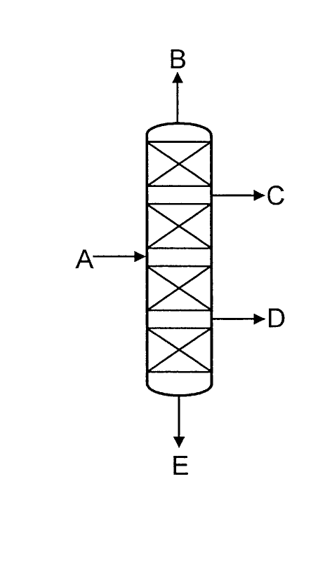

A mixture of 20.6% by weight chlorobenzene, 5.2% by weight n-butyl α-isocyanopropionate (R₁=n-butyl, R₂=methyl) and 72.60% by weight tris(2-ethylhexyl)amine was continuously fed into a column as in Example 1, but without dividing wall (see FIG. 1) through inlet (A).

With an overhead pressure of 300 mbar and a bottom temperature of 165° C., the solvent is taken off at the top (B). The 4-methyl-5-n-butoxyoxazole is obtained in a yield of 99% through the side offtake (C). The amine is discharged through the bottom offtake (E).

### EXAMPLE 4

Continuous preparation of 4-methyl-5-n-butoxyoxazole a reaction column

As in Example 3, a mixture of 13.14% n-butyl α-isocyanopropionate and 86.86% tris(2-ethylhexyl)amine is continuously fed in through inlet (A).

With an overhead pressure of 400 mbar and a bottom temperature of 165° C., the 4-methyl-5-n-butoxyoxazole is taken off at the top (B) and the amine is discharged through the bottom offtake (E). The yield of 4-methyl-5-n-butoxyoxazole was 98.8%.

### EXAMPLE 5

Continuous preparation of 4-methyl-5-isobutoxyoxazole in a reaction column

As in Example 3, a mixture of 22.7% isobutyl α-isocyanopropionate and 77.3% N,N-dibutylaniline is continuously fed in through inlet (A).

With an overhead pressure of 300 mbar and a bottom temperature of 160° C., the 4-methyl-5-isobutoxyoxazole is taken off at the top (B) at a temperature of 150° C. The amine is obtained at 161° C. through the side offtake D. The yield of 4-methyl-5-isobutoxyoxazole is 91%.

### EXAMPLE 6

Continuous preparation of 4-methyl-5-n-butoxyoxazole in a reaction column

As in Example 5, a mixture of 11.8% n-butyl α-isocyanopropionate and 88.2% N,N-dibutylaniline is continuously fed in through inlet (A). 4-Methyl-5-n-butoxyoxazole is taken off in a yield of 98.7% at the top (B), and the amine is obtained through the side offtake D.

We claim:

1. A process for the continuous preparation of 5-alkoxy-substituted oxazoles of the formula I, 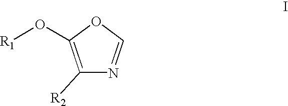
where
R₁ is an optionally substituted C₁-C₆-alkyl radical and
R₂ is hydrogen or an optionally substituted C₁-C₆-alkyl radical,
by converting α-isocyanoalkanoic esters of the formula II 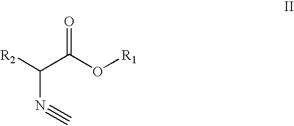
which are fed in continuously, in the presence of bases which are fed in continuously, at temperatures above 80° C. in a reactor into the 5-alkoxy-substituted oxazoles of the formula I and discharging the reaction products continuously from the reactor.

2. A process as claimed in claim 1, wherein the 5-alkoxy-substituted oxazoles of the formula I are removed from the reaction mixture simultaneously with the conversion.

3. A process as claimed in claim 1 or 2, wherein a reaction column is used as reactor, and the 5-alkoxy-substituted oxazoles of the formula I are removed from the reaction mixture by rectification simultaneously with the conversion.

4. A process as claimed in claim 3, wherein the rectification parameters are adjusted so that
A the conversion of the α-isocyanoalkanoic esters of the formula II into the 5-alkoxy-substituted oxazoles of the formula I takes place on the internals and, where appropriate in the bottom of the reaction column,
B the 5-alkoxy-substituted oxazoles of the formula I produced in the conversion are removed continuously with the overhead stream or side stream of the reaction column and
C the base, and the high boilers produced where appropriate in the conversion, are removed continuously and independently of one another with the bottom stream or side stream of the reaction column.

5. A process as claimed in any of claims 1 to 4, wherein the conversion is carried out in the presence of an inert solvent, and the reaction parameters are adjusted so that
A the conversion of the α-isocyanoalkanoic esters of the formula II into the 5-alkoxy-substituted oxazoles of the formula I takes place on the internals and, where appropriate in the bottom of the reaction column,
B1 in the case where the solvent has a higher boiling point than the 5-alkoxy-substituted oxazoles of the formula I produced in the conversion, the 5-alkoxy-substituted oxazoles of the formula I are removed continuously with the overhead stream, and the solvent is removed continuously via the side stream or bottom stream of the reaction column, and
B2 in the case where the solvent has a lower boiling point than the 5-alkoxy-substituted oxazoles of the formula I produced in the conversion, the 5-alkoxy-substituted oxazoles of the formula I are removed continuously with a side stream, and the solvent is removed continuously with the overhead stream of the reaction column, and
C the base, and the high boilers produced where appropriate in the conversion, are removed continuously and independently of one another with the bottom stream or side stream of the reaction column.

6. A process as claimed in any of claims 3 to 5, wherein a dividing wall column is used as reaction column.

7. A process as claimed in any of claims 3 to 6, wherein in the case where the base forms an azeotrope with the 5-alkoxy-substituted oxazoles of the formula I, the overhead pressure in the column is adjusted so that the proportion of base in the azeotrope in the overhead stream is minimized.

8. A process as claimed in any of claims 4 to 7, wherein the overhead pressure of the column is adjusted to 5 to 800 mbar, and the bottom pressure which results therefrom, depending on the type of column used and, where appropriate, the type of column internals used, is 10 mbar to atmospheric pressure.

9. A process for preparing pyridoxine derivatives of the formula IX 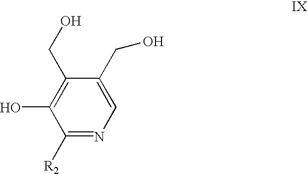
where
R₂ is hydrogen or an optionally substituted C₁-C₆-alkyl radical,
by converting amino acids of the formula III 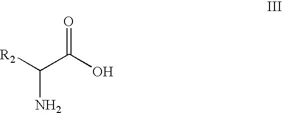
into amino acid esters of the formula IV, 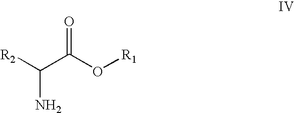
where
R₁ is an optionally substituted C₁-C₆-alkyl radical,
converting the latter into formamido acid esters of the formula V, 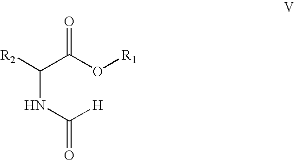
converting the latter into α-isocyanoalkanoic esters of the formula II 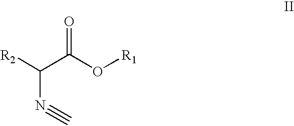
converting the latter in a continuous process step in the presence of bases at temperatures above 80° C. into 5-alkoxy-substituted oxazoles of the formula I, 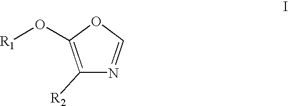
reacting the latter with protected diols of the formula VI, 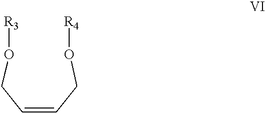
where
R₃, R₄ are, independently of one another, or R₃ and R₄ together, are a protective group of the hydroxyl function,
to give the Diels-Alder adducts of the formula VII 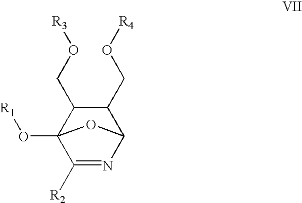
and converting the latter by acid treatment and elimination of the protective group into the pyridoxine derivatives of the formula IX.
